(19) United States
(12) Patent Application Publication
(10) Pub. No.: US 2019/0252078 A1
(43) Pub. Date: Aug. 15, 2019

(54) PREDICTING THE SPREAD OF CONTAGIONS

(71) Applicant: X Development LLC, Mountain View, CA (US)

(72) Inventors: Martin Friedrich Schubert, Mountain View, CA (US); Adam Sadilek, San Jose, CA (US)

(21) Appl. No.: 15/897,324

(22) Filed: Feb. 15, 2018

(51) Int. Cl. G16H 50/80; G16H 50/70; G16H 10/60; G06F 15/18

(52) U.S. Cl. CPC G16H 50/80; G06F 15/18; G16H 10/60; G16H 50/70

(57) ABSTRACT

Methods, systems, and apparatus, including computer programs encoded on a computer storage medium, for obtaining internet search data, the search data indicating internet searches performed by a population of users. Obtaining location data associated with each user in the population where the location data represents one or more geographic locations of each user over a period of time. Identifying a subset of the population who are likely carrying a contagion based on the search data. Determining an exposure level of a user to the contagion based on a correlation of a first location data associated with the user with a second location data associated with one or more users in the subset of the population who are likely carrying the contagion. Determining whether the user is likely to be or become ill based on the exposure level. Providing a notification indicating that the user has been exposed to the contagion.

### TECHNICAL FIELD

This disclosure generally relates to predicting the spread of contagious disease.

### BACKGROUND

Predicting the spread of contagious disease is important to public health and safety. Present modeling techniques for estimating the spread of contagious diseases rely heavily on human intervention and reporting from hospitals and private practices. Furthermore, present modeling techniques can only predict disease spread on a regional level. The present techniques are not capable of predicting the spread of a disease at an individual-to-individual level.

### SUMMARY

In general, the disclosure relates to a machine learning system that uses internet search data and individual location data to predict the spread of illness on an individual-to-individual basis. The system can further perform macroscopic predictions based on multiple individual predictions. More specifically, the system identifies subsets of individuals from a population who are likely carrying a contagion based on internet search data (e.g., identifying individuals who have recently searched for information about the contagion). The system correlates location data for each member of the population to identify members of the population who have been exposed to the potential contagion carriers. The system can predict a likelihood that any individual will become affected by the contagion based the correlation.

For example, a machine learning system can use a combination of internet search data and individual user location data to predict whether a unique individual will become ill. The system can identify a subset of users out of a population who are likely carrying a contagion (e.g., a virus) based on internet search data. The system can identify internet search data that includes, for example, internet search logs that indicate topics of individual websites describing symptoms of an illness, which websites a user viewed, and how long the user viewed the website. The system can use a machine learning model to process the search data to identify users that are likely ill or carrying a contagion based searching trends indicated in the search logs.

The system can determine exposure levels for individual users by correlating location data of the individual users with location data of the users in the potentially contagious subset. For example, a user whose location is correlated to that of a potentially contagious user within a specified timeframe has likely been exposed to the contagion. The system can determine an exposure level for each individual based on the number of times that the individual has crossed paths with a potentially contagious user and the length of each exposure. The system can predict a likelihood that each individual user will become ill based on their exposure level.

In general, innovative aspects of the subject matter described in this specification can be embodied in methods that include the actions of obtaining internet search data, the search data indicating internet searches performed by a population of users. Obtaining location data associated with each user in the population where the location data represents one or more geographic locations of each user over a period of time. Identifying a subset of the population who are likely carrying a contagion based on the search data. Determining an exposure level of a user to the contagion based on a correlation of a first location data associated with the user with a second location data associated with one or more users in the subset of the population who are likely carrying the contagion. Determining whether the user is likely to be or become ill based on the exposure level. Providing, for display on a user computing device, a notification indicating that the user has been exposed to the contagion.

Other implementations of this aspect include corresponding systems, apparatus, and computer programs, configured to perform the actions of the methods, encoded on computer storage devices. These and other implementations can each optionally include one or more of the following features.

Some implementations include determining a trend in a spread the contagion based on aggregating predictions for a plurality of individual users.

Some implementations include identifying an action for avoiding exposure to the contagion, and providing, to a computing device associated with the user, a notification alerting the user to the action.

In some implementations, determining the exposure level of the user to the contagion includes determining that the user was present in a geographic region within an exposure window.

Some implementations include obtaining environmental data for the geographic region, wherein the exposure window for the geographic region is based, at least in part, on the environmental data.

In some implementations, determining the exposure level of the user to the contagion includes using a geographic grid to compare the first location data with the second location data.

In some implementations, determining the exposure level of the user to the contagion includes using a semantic map to compare the first location data with the second location data.

In some implementations, identifying the subset of the population who are likely carrying by the contagion comprises identifying a class of the contagion.

In some implementations, the internet search data includes internet search logs.

In some implementations, the internet search logs include one or more annotations indicating topics described in search results, topic weightings, and an amount of time a user spent viewing one or more of the search results.

Some implementations include obtaining additional user information, wherein identifying the subset of the population who are likely carrying the contagion comprises identifying the subset based on the internet search data and the additional user information.

In some implementations, the additional user information includes one or more of: user voice data, user biometric data, user motion data, or data indicating changes in a user's routine.

Particular implementations of the subject matter described in this specification can be implemented so as to realize one or more of the following advantages. Implementations may provide improvements in prediction accuracy over existing disease modeling technologies. For example, existing modeling technologies are not capable of generating individualized predictions. For example, existing modeling techniques cannot predict whether a unique individual in a population will become ill, but only provide predictions on the spread of a disease across a broad region. Implementations protect users' privacy by eliminating human interactions with data. For example, implementations employ machine learning techniques and data gathering rules that permit a computer system to generate individualized predictions of data flow without the need for human interactions. For example, implementations can collect search result data based on annotations in search logs that indicate website topics included in the search.

The details of one or more implementations of the subject matter of this disclosure are set forth in the accompanying drawings and the description below. Other features, aspects, and advantages of the subject matter will become apparent from the description, the drawings, and the claims.

### DESCRIPTION OF DRAWINGS

FIG. 1 depicts block diagram of an example system for predicting the spread of contagions.

FIG. 2 depicts a graphical representation of exemplary processes for determining exposure levels for individual users.

FIG. 3 depicts a chart showing correspondence between experimental results and actual diagnoses.

FIG. 4 depicts a flowchart of an example process for predicting the spread of contagions in accordance with implementations of the present disclosure.

FIG. 5 depicts a schematic diagram of a computer system that may be applied to any of the computer-implemented methods and other techniques described herein.

Like reference numbers and designations in the various drawings indicate like elements.

### DETAILED DESCRIPTION

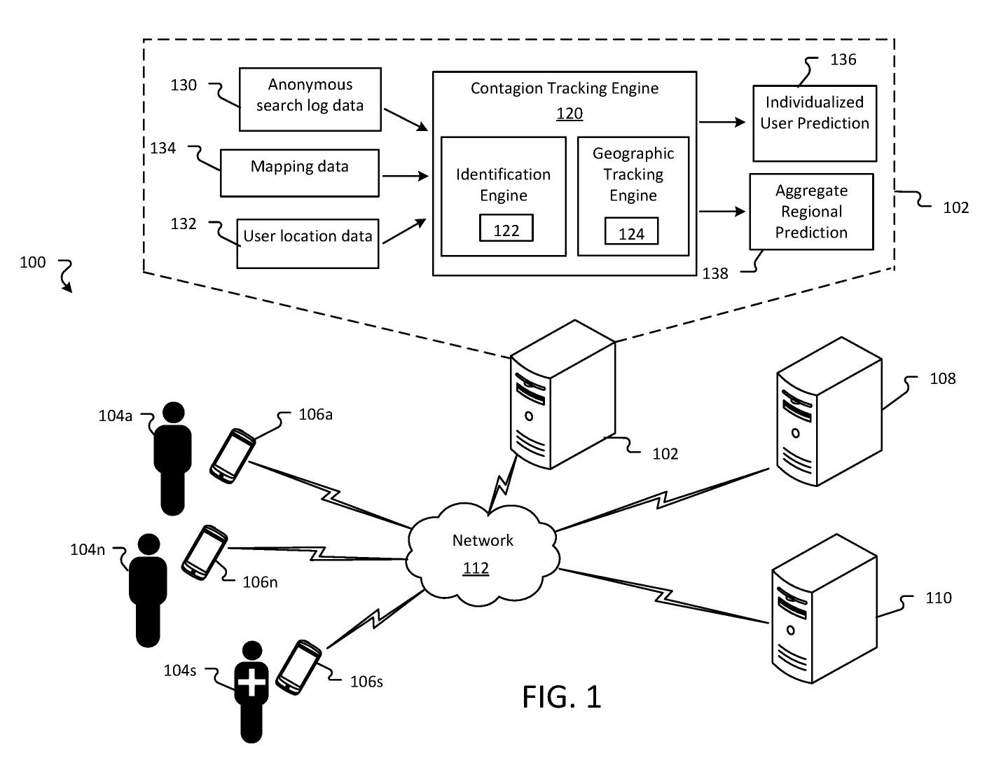

FIG. 1 is a diagram that illustrates an example of a system 100 for predicting the spread of contagions. The system 100 includes a server system 102 in communication with a plurality of user devices 106a-106n, 106s (collectively 106) one or more search systems 108, and one or more mapping systems 110. Server system 102 communicates with user devices 106, search system 108 and mapping system 110 over a network 112. Network 112 can include public and/or private networks and can include the Internet. In some implementations, the system 100 includes non-user devices (not shown) such as, but not limited to, environmental sensors, air quality sensors, and occupancy sensors.

The server system 102 can include a system of one or more computers. The server system 102 is configured to predict the spread of an illness based on individualized user information and location data. For example, the server system 102 can store and execute one or more machine learning engines that are programmed to predicted the likelihood that any individual will become ill by performing the processes described below. For example, the server system 102 can include data access rules that enable the server system 102 to anonymously obtain user information, as described herein, that is indicative of whether a user (e.g., user 104s) is ill. The server system 102 can obtain the user information from user devices 106, search systems 108, or both. The server system 102 also obtains user location data from user devices 106. In some implementations, the data access rules permit server system 102 to obtain the user information and/or location data without human interaction, thereby, protecting the users' privacy.

The server system 102 can also protect each user's privacy by providing a contagion tracking application (e.g. a downloadable application or web-based application). For example, the server system 102 can permit users to opt-in or opt-out of having their information used for contagion tracking. In some implementations, the server system 102 can assign anonymous identification credentials to each user whose data is obtained. The server system 102 can use the anonymous identification credentials to correlate data to specific users to protect personal information. Furthermore, the processes described below for obtaining and analyzing each user's data to track contagions are designed to be executed automatically by the server system 102 such that human intervention is not required. For instance, the server system 102 can be configured to use data access rules and machine learning models to predict the spread of a disease on an individual basis without human intervention and to protect each individual's privacy.

User devices 106 can be computing devices, e.g., mobile phones, smart phones, tablet computers, wearable computing devices, laptop computers, desktop computers, home assistant devices, or other portable or stationary computing device. User devices 106 can feature a microphone, keyboard, touchscreen, speaker, or other interfaces that enable users 104a-n, 104s (collectively 104) to provide inputs to and receive output from user device 106a. User device 106a can include a camera, accelerometers, GPS receiver, and/or other sensors that enable user devices 106 to obtain information about the surrounding environment and the location of user device 106, and, by extension, the users 104.

The search system 108 can be a server or a network of servers that hosts and executes a search engine (e.g., in internet search engine). The search system 108 can be a third-party system, operated independently of the server system 102. The server system 102 can communicate with multiple search systems 108, and each may execute a different search engine.

The search system 108 creates and stores logs of the searches performed by users. In some implementations, the search logs include annotations that provide details of individual searches performed by the search system 108. For example, the search logs can include annotations that provide, but are not limited to, topics described in each search result (e.g., webpage), topic weightings (e.g., weighting that indicate the relative importance of each topic within a specific search result), an amount of time a user spent viewing each search result, indications of which web pages were clicked or read, and the location from which the query was issued.

The mapping system 110 can be a server or a network of servers that hosts and executes a mapping engine. The mapping system 110 can be a third-party system, operated independently of the server system 102. The server system 102 can communicate with multiple mapping system 110, and each may execute a different mapping engine. The mapping system 110 creates and stores digital maps of geographic regions. In some implementations, the mapping system 110 provides semantic maps. A semantic map is a digital map that references geographic locations using semantic title. For example, a semantic map can correlate geographic location data (e.g., GPS coordinates) to geographic locations using a semantic title (e.g., the latitude and longitude of the White House can be correlated with the name “White House”). In some implementations, a semantic map can correlate a geographic region to a semantic title. For example, title “White House” can be correlated to the GPS coordinates that define the geographic footprint of the actual White House. In some implementations, semantic mapping can be used to determine when a person is located inside a building and which specific building. In some implementation, a semantic map can resolve geographic regions down to specific rooms within a building. For example, a semantic map can distinguish between the locations of different stores within a mall and label the respective geographic regions with the names of the stores.

In various implementations, server system 102 can perform some or all of the operations related to predicting the spread of contagions. For example, server system 102 can include a contagion tracking engine 120. Contagion tracking engine 120 can implement the data access rules for anonymously obtaining user information and location data. Contagion tracking engine 120 can also implement one or more machine learning engines that analyze the user information and location data to generate individualized predictions regarding the spread of a contagion. For example, contagion tracking engine 120 can include an identification engine 122 and a geographic tracking engine 124. Identification engine 122 and geographic tracking engine 124 can be implemented as separate machine learning engines or as two modules of one machine learning engine.

More specifically, contagion tracking engine 120 includes one or more machine learning models that have been trained to receive model inputs (e.g., user information such as anonymous search log data and location data such as GPS data associated with a subset of registered users) and to generate a predicted output (e.g., predictions of one or more users who are likely are or will become ill) based on the received model input. In some implementations, the machine learning model is a deep model that employs multiple layers of models to generate an output for a received input. For example, the machine learning model may be a deep neural network. A deep neural network is a deep machine learning model that includes an output layer and one or more hidden layers that each apply a non-linear transformation to a received input to generate an output. In some cases, the neural network may be a recurrent neural network. A recurrent neural network is a neural network that receives an input sequence and generates an output sequence from the input sequence. In particular, a recurrent neural network uses some or all of the internal state of the network after processing a previous input in the input sequence to generate an output from the current input in the input sequence. In some other implementations, the machine learning model is a shallow machine learning model, e.g., a linear regression model or a generalized linear model.

In operation, contagion tracking engine 120 obtains user information such as anonymous search log data 130 from search system 108. For example, contagion tracking engine 120 can obtain internet search logs associated with a population of users 104. The search logs may contain user search data including, but not limited to, topics described in each search result, topic weightings, and an amount of time a user spent viewing each search result. In some implementations, the search logs can contain information that identifies a unique user in an anonymized manner. For example, the search logs may include user ID information from if the user performed the search while signed-in to a user account (e.g., an associated email account). As another example, the search logs may include identification information from cookie-based id's. The search logs may be a less intrusive way of obtaining user search information than using search queries, for example, because they provide results generated by the search engine and not information entered by a user. The accuracy with which the machine learning models process the user information can be improved by the use of search logs instead of search query data.

Contagion tracking engine 120 obtains user location data 132. User location data can include, for example, GPS data, WiFi location data, or cellular location data. For example, contagion tracking engine 120 can obtain user location data from user computing devices 106 (e.g., mobile devices) or from location data logs. As discussed in more detail below, contagion tracking engine 120 may only obtain user location data from user devices 106 that are associated with users who have “opted-in” to the contagion tracking system by, for example, downloading an associated mobile application.

Identification engine 122 processes the user information to estimate a health state of users represented by the user information. For example, identification engine 122 can identify individual users (e.g., 104s) who are likely ill or carrying a contagion based on the search log data 130. Identification engine 122 can identify indications that a user may be ill within the search log data. Search log data 130 that provide indications that a user may be ill can include, but are not limited to, topics described in each search result, topic weightings, and amounts of time a user spent viewing a search result. Identification engine 122 can predict based on the search log annotation data whether a unique user is likely ill. Identification engine 122 can then designate a user ID of each user who is identified as likely ill as a potentially infected user 104s. Those users 104s designated as potentially infected may represent a subset of users who are likely carrying a contagion from among a population of users 104. For example, each user can be assigned a probability value of being ill at the time of the search. In some implementations, the probability can be predicted into the future.

For example, search log annotations indicating that user 104s spent an hour viewing several webpages discussing flu symptoms and treatment may provide a strong indication that the user the flu or flu-like symptoms. Identification engine 122 can designate that user 140s as likely carrying a contagion. By contrast, identification engine 122 can view search log annotations indicating that user 104a spent ten minutes viewing a webpage discussing news about flu vaccines either irrelevant to whether user 104a is ill or as a weak indication that user 104a is ill. In response, identification engine 122 would not designate user 104a as likely carrying a contagion. Moreover, users 104a and 104s may have entered the same or a similar search query to obtain their search results (e.g., “flu information”). However, by using the search log data 130 to identify potentially ill users 104 instead of (or in addition to) search query data, identification engine 122 can more accurately identify users who may be carrying a contagion.

Geographic tracking engine 124 determines exposure levels for non-infected users (e.g., users 104a-104n who have not been identified by identification engine 122 as being potentially infected). Geographic tracking engine 124 can use the location data associated with both infected and non-infected users to determine how often each user has been exposed to potentially infected users 104s. For example, geographic tracking engine 124 receives indications of potentially infected users from identification engine 122 and correlates user location data of the potentially infected users 104s with that of the non-infected users 104a-104n to determine exposure levels for the non-infected users 104a-104n. For example, if location data for users 104a and 104s indicate that non-infected user 104a was present within the same predefined geographic region as potentially infected user 104s within the same time period, geographic tracking engine 124 can increase an exposure level associated with user non-infected 104a. Geographic tracking engine 124 can determine an exposure level for each user 104 based on exposure factors including, but not limited to, the number of exposures each user has with potentially infected users, the duration of time that each user was exposed to a potentially infected user, the elapsed time between exposures to potentially infected users, or a combination thereof. The exposure level can be incrementally increased for each exposure a non-infected user has with a potentially infected user according to the exposure factors associated with each exposure. For example, an exposure level can be represented as a value within a range of 0 to 100 “exposure points,” with zero representing no exposures to potentially infected users. An exposure that lasts only a short duration (e.g., 1 exposure point) may increase the exposure level by a smaller increment that one that lasts for a longer duration (e.g., 5 exposure points).

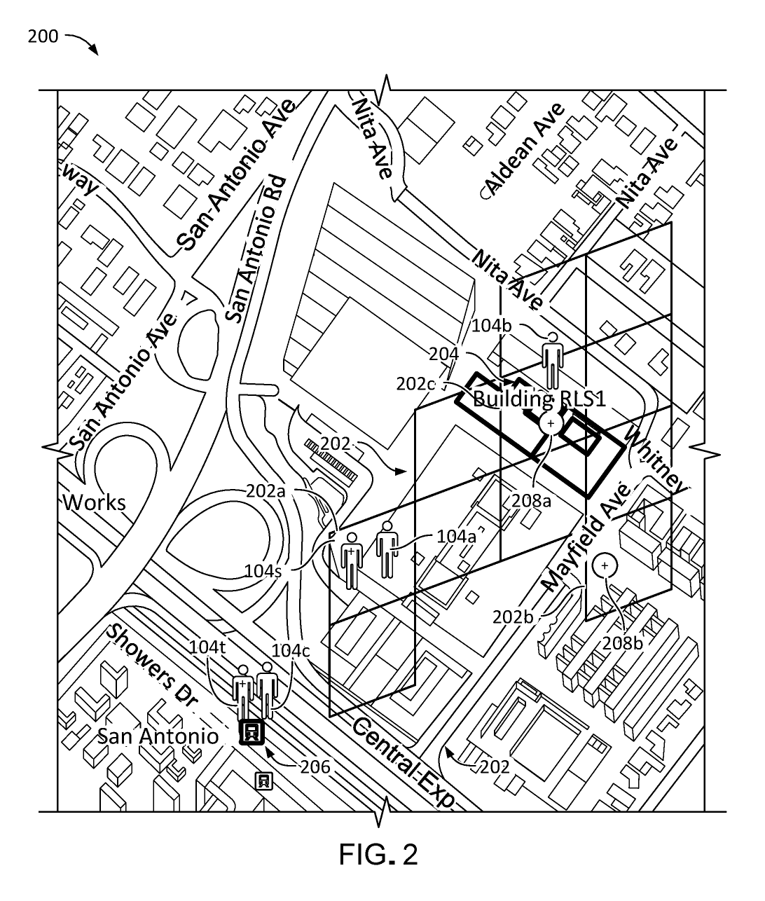

In some implementations, contagion tracking engine 120 can obtain mapping data 134 from a mapping system 110. Geographic tracking engine 124 can use the mapping data 134 to determine user exposure levels. For example, FIG. 2 depicts a graphical representation 200 of exemplary processes for determining exposure levels for individual users 104 that incorporate mapping data 134.

One example process uses a geographic grid 202. Grid 202 divides a geographic region into a plurality of geographic cells. Geographic tracking engine 124 can determine exposure levels for individual users 104a-104n by identifying whether, based on a user location data, a non-infected user 104 was located within the same cell (e.g., cell 202a) with a potentially infected user 104s within a predetermined exposure window. For example, the exposure level for user 104a exposure level can be increased if user 104a was located within cell 202a at the same time as user 104s.

Another example process uses a semantic map. As noted above, a semantic map is a digital map that references geographic location (e.g., semantic regions 204, 206) using semantic titles, 206. For example, “Building RLS1” is a semantic region 204 representing the geographic area occupied by the actual Building RLS1. Similarly, “San Antonio” is a semantic region 206 representing the geographic area occupied by the actual San Antonio Ave. bus stop. Geographic tracking engine 124 can determine exposure levels for individual users 104a-104n by identifying whether, based on a user location data, a non-infected user was located within the same semantic region with a potentially infected user within a predetermined exposure window. For example, the exposure level for user 104a exposure level can be increased if user 104c was located in region 206 (e.g., San Antonio bus stop) at the same time as potentially infected user 104t.

In some implementations, the geographic tracking engine 124 can implement a hybrid process that uses both types of mapping data 134: geographic grids 202 and semantic maps. For example, geographic tracking engine 124 can use a geographic grid 202 to divide outdoor areas into exposure zones and, thereby, determine user exposure levels in outdoor areas. Geographic tracking engine 124 can use the semantic regions of a semantic map to determine user exposure levels in indoor areas and/or well-defined outdoor areas, such as an outdoor bus stop.

In some implementations, geographic tracking engine 124 can establish an exposure window for each cell. An exposure window is a period of time during which pathogens from a potentially infected user 104s may be present within a given geographical region (e.g., a grid cell or semantic region) after a potentially infected user 104s leaves the region. For example, a flu virus may be present and contagious within a given from when a potentially infected user 104s arrives in the region and may remain contagious for a period of time after the user 104s leaves the region. The exposure window accounts for the time that the pathogen remains even after the potentially infected user 104s leaves the region. For example, a flu virus may remain contagious within a region for an hour after a potentially infected user 104s leaves. Therefore, geographic tracking engine 124 can establish an exposure window for each geographic region that extends for one hour longer than the time that a potentially infected user 104s was located in the region. For example, icon 208a indicates that cell 202c (or Building RLS1) is within an exposure window and presents an exposure risk to user 104b even though a potentially infected user is not present at the same time.

Geographic tracking engine 124 can predict a likelihood that users 104 will become ill based on their exposure levels. For example, geographic tracking engine 124 can identify a user 104a as being likely to become ill if the user's exposure level exceeds a threshold exposure value. That is, if a user 104a is sufficiently exposed to contagious users, the user is more likely to become ill. For example, geographic tracking engine 124 can compare each user's exposure level to the threshold exposure value to determine which individual users are likely to become ill. Moreover, each user can be assigned a probability value of being ill based on the comparison. For instance, a user's probability of becoming ill may increase in relation to the amount by which the user's exposure level exceeds the threshold value. In some implementations, geographic tracking engine 124 can compare each user's exposure level to the threshold exposure value at regular intervals. In some implementations, geographic tracking engine 124 can compare each user's exposure level to the threshold exposure value when the exposure level changes.

Contagion tracking engine 120 can generate individualized user predictions 136 for users that geographic tracking engine 124 identifies as likely to become ill. For example, contagion tracking engine 120 can send a notification (e.g., a “sickness notification”) to a user 104a to inform the user that he has been exposed to a pathogen and will likely become ill within a certain period of time. The sickness notification can include, but is not limited to, a mobile application notification, an SMS message, an e-mail, or any combination thereof. Contagion tracking engine 120 then transmits the notifications to user devices 106 associated with the users who have been identified as likely to become ill. For example, the threshold exposure value for determine whether a single individual will become ill due to exposure to potentially infected users may be set such that users can be notified within an incubation period of the illness, thereby, permitting users to take preventative actions (e.g., getting a vaccination, taking immune system boosting vitamins, etc.). For example, in some implementations, the threshold exposure value can be adaptable based on feedback to the machine learning algorithm. For example, the threshold exposure value can be set such that users who are likely to become ill can be informed within sufficient time to take preventative actions to avoid becoming sick.

In some implementations, contagion tracking engine 120 can provide preemptive notifications to individuals. A preemptive notification can be sent to a single user's user device 106 in order to prevent the user from being exposed to a potentially infected user. For example, based on user location data, the contagion tracking engine 120 can identify an action that a non-infected user can take to avoid an exposure to a potentially infected user. Contagion tracking engine 120 can transmit a preemptive notification information the non-infected user of how to avoid the exposure. Moreover, contagion tracking engine 120 can issue such preemptive notification without violating the privacy of either the non-infected or the infected user because no human intervention is required and the notification need only suggest an action without identifying the potentially infected individual. For example, if contagion tracking engine 120 detects, based on user location data, that non-infected user 104a is waiting at San Antonio bus stop and that that a potentially infected user 104s is on the next bus to arrive at the bus stop, contagion tracking engine 120 can send a notification to the user's user device 106a informing the user to take another bus. In other words, a preemptive notification can inform a non-infected user to adjust their behavior in order to avoid exposure to a pathogen.

In addition to or in lieu of individualized user predictions, contagion tracking engine 120 can generate one or more aggregate predictions 138. For example, contagion tracking engine 120 can aggregate individual user predictions associated with users located in defined geographic region to generate a regional prediction. The aggregate prediction may represent a potential outbreak of a type of illness or type of illness. For example, contagion tracking engine 120 may identify an increase in the number of users whose exposure level exceeds the exposure threshold within a city. Contagion tracking engine 120 can transmit appropriate illness outbreak information to a local authority, such as a government heath office, a hospital, or the Center for Disease Control (CDC), regarding the aggregate predictions. For example, contagion tracking engine 120 may inform hospitals in within a certain city. In some implementations, contagion tracking engine 120 can determine the number of potentially infected people over time, the geographic extent over which the contagion has spread or may spread, an estimated presymptomatic incubation period, an average length of an infection, a estimated post symptomatic infectiousness, the infectiousness of the disease, an estimate of how the contagion spreads (e.g., by contact or airborne), or a combination thereof.

Moreover, because the contagion tracking engine 120 can track the actual movement of individuals, the contagion tracking engine 120 can also predict a geographic spread of for an outbreak of a type of illness. In other words, the contagion tracking engine 120 can aggregate the actual movements of individuals-both those who are determined to be likely infected and those whose exposure levels indicate they will likely become infected—to detect and predict regional trends for how an outbreak of a disease may spread. By using the actual user location data, the contagion tracking engine 120 can determine both intensity and direction that a disease may spread. For instances, the intensity of spread may be represented by the total number of potentially infected users and users who are likely to become infected that travel in a given direction (e.g., commute to the same city or return to the same suburb). And the direction of spread is represented by the direction that users, as an aggregate, travel. Thus, contagion tracking engine 120 may transmit information related to the predicted spread of an outbreak to authorities in surrounding regions to halt emerging epidemics before they become pandemic.

In some implementation, user information used to identify potentially infected individuals can include, but is not limited to, user voice data, user biometric data, user motion data, and data indicating changes in a user's routine. For example, user voice data may indicate that a user coughing or sneezing while providing voice commands to a home assistant device. Detection of a cough or sneeze may indicate that the user is ill or may corroborate search log data indicating that the user is ill. As another example, changes in user biometric data (e.g., heart rate, sleep patterns, etc.) from a wearable device may indicate that the user is ill or may corroborate search log data indicating that the user is ill. As another example, changes in user motion data (e.g., lethargic movements) from a wearable device may indicate that the user is ill or may corroborate search log data indicating that the user is ill. As another example, changes in data indicating changes in a user's routine (e.g., location data indicating that a user visited a doctor's office or that a user stayed home from work or school) may indicate that the user is ill or may corroborate search log data indicating that the user is ill.

In some implementations, the identification engine 122 can classify illnesses based on the user information (e.g., search log data, voice data, biometric data, etc.). For example, identification engine 122 can group users who are identified as likely ill based on a class of illness that the users are likely carrying. For example, illness classes can include, but are not limited to, upper respiratory diseases, flu-like diseases, food poisoning, or a particular disease (e.g., Lyme disease). For example, a first set of search logs may indicate that one user has been searching for symptoms of Lyme disease. Identification engine 122 can identify that user as being likely ill with or carrying Lyme disease. At the same time, a combination of user voice data (e.g., coughing) and search log data may indicate that another user has symptoms of an upper respiratory disease. Identification engine 122 can identify the second user as being likely ill with or carrying an upper respiratory disease.

In some implementations, contagion tracking engine 120 can incorporate different machine learning models to track the spread of different classifications of illnesses. For example, geographic tracking engine 124 may include illness specific machine learning models such as one machine learning model to track upper respiratory illnesses and another to track food poisoning. In some implementations, diseases having similar properties may be tracked using the same machine learning model. For example, diseases with similar length incubation periods may be track using one machine learning model.

In some implementations, geographic tracking engine 124 can use disease attributes to adjust tracking parameters. For example, geographic tracking engine 124 can adjust an exposure window for a particular disease based on how long the respective pathogen can survive. In some implementations, geographic tracking engine 124 can also incorporate environmental data associated with each geographic region. For example, a given pathogen may be more contagious in a dry environment than in a humid environment or may survive longer in a dry environment compared to a humid environment. Geographic tracking engine 124 can adjust the length of the exposure window in a particular region based on the pathogen's contagiousness or survivability in view of existing environmental conditions. For example, an exposure window for cell 202c which is inside Building RLS1 may be longer than the exposure window for cell 202b which is outside in a parking lot. In some implementations, contagion tracking engine 120 can obtain environmental information from outside sources such as weather stations for outdoor locations and smart devices (e.g., a smart thermostat) for indoor locations. Geographic tracking engine 124 can incorporate the environmental information into a machine learning model to adjust the exposure windows for different geographic regions.

In some implementations, contagion tracking engine 120 can vary the timing with which it generates individualized user predictions 136 for users that the geographic tracking engine 124 identifies as likely to become ill. For example, implementations in which the contagion tracking engine 120 differentiates between different classes of illnesses can generate predictions such that a sickness notification can be sent to affected users early enough to permit the users to prevent the illness. For instance, different classes of illnesses may have different incubation periods during which an infected individual could prevent the illness from reaching its full effect. In some implementations, geographic tracking engine 124 can account for such differences in incubation period by adjusting the exposure threshold values accordingly for different classes of illnesses. For example, geographic tracking engine 124 may accommodate for an illness that has a relatively short incubation period by reducing the associated exposure threshold value. Reducing the threshold value would permit the geographic tracking engine 124 to identify users who might become ill sooner, thereby, allowing the contagion tracking engine 120 to transmit appropriate sickness notifications earlier so the affected users can take appropriate precautions. As another example, geographic tracking engine 124 may accommodate for an illness that has a relatively long incubation period by increasing the associated exposure threshold value. Increasing the threshold value may reduce the number of false positive predictions, while still providing sufficient time for the contagion tracking engine 120 to transmit appropriate sickness notifications to the affected users in time to take appropriate precautions.

In some implementations, contagion tracking engine 120 can model people in a population who are not registered users by estimating the effects of a contagion on such non-users. For example, contagion tracking engine 120 can employ simulated agents who have realistic movements and schedules that approximate non-users. As another example, contagion tracking engine 120 can use other proxy information to account for non-users such as the number of passengers and schedule of public transit or flights to estimate movements of non-users and exposures of non-users with potentially infected users. For example, part of the population that are non-users can be added to the model as artificial agents with simulated behaviors consistent with known statistics about human movement and activities, such as, but not limited to following distributions of mobility and/or commute patterns and regularization from an America Time Use Survey and/or a census.

In some implementations, the machine learning model(s) of the contagion tracking engine 120 are continually retrained. For example, the contagion tracking engine 120 can provide surveys to users who have been identified as likely to become ill. Contagion tracking engine 120 can provide the surveys through an associated application. The survey can be used to obtain prediction validation information by requesting users to indicate whether they became sick. For example, contagion tracking engine 120 can receive survey results from user inputs to their respective user devices 106. Contagion tracking engine 120 can train the machine learning models using the survey results. For example, the machine learning models can use the validation information from the survey results to adjust model parameters such as exposure thresholds and exposure windows. In some implementations, the machine learning models can be trained using labeled datasets that have been generated by a separate machine learning model.

Contagion tracking engine 120 is configured to protect user information (e.g., search log information and location data) and privacy. For example, data access rules can prohibit the contagion tracking engine 120 from processing search information from unknown MAC addresses. For example, a user may opt-in to the contagion tracking system by downloading an application and establishing a user ID. The contagion tracking engine 120 can identify search log data and/or location data associated with MAC addresses of computing devices registered to user ID's of users who have provided permission to do so by “opting in” to the contagion tracking system. In addition, data access rules can prohibit the contagion tracking engine 120 from obtaining user location data from mobile devices of users who have not downloaded the associated application or have not “opted in” to the contagion tracking system. The contagion tracking engine 120 can protect user privacy by encrypting user IDs, encrypting MAC addresses, removing any private information from the search logs, or a combination thereof.

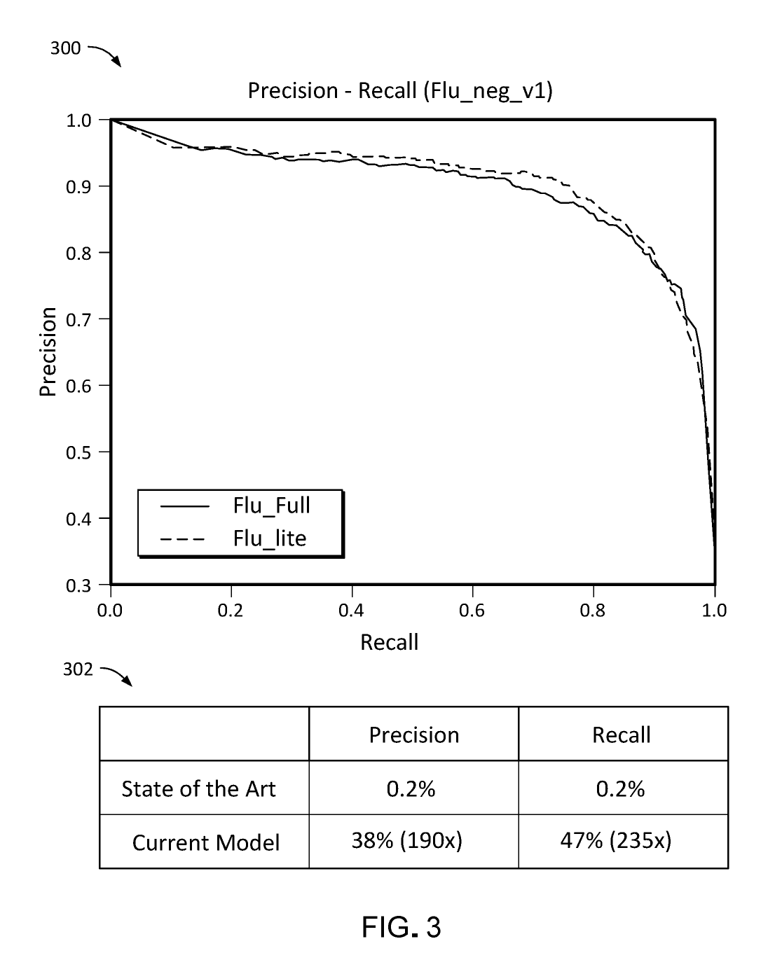

FIG. 3 depicts a graph 300 and a chart 302 showing experimental results obtained from machine learning models executing the above described processes. Graph 300 shows the correspondence between experimental results and actual diagnoses. Machine learning model predictions of a likelihood that a user is ill based on search log data were compared with judgements made by physicians. The machine learning model predictions exhibited a close correlation with the judgements of the physicians. Furthermore, recent versions of the machine learning model have been compared to survey's supplied by test users. In the experiments, the machine learning models predicted the likelihood that individual users would become ill with a 38% precision and a 47% recall, as shown in chart 302. Furthermore, these results represent a 190 times improvement in prediction precision over and a 235 times improvement over prior prediction systems.

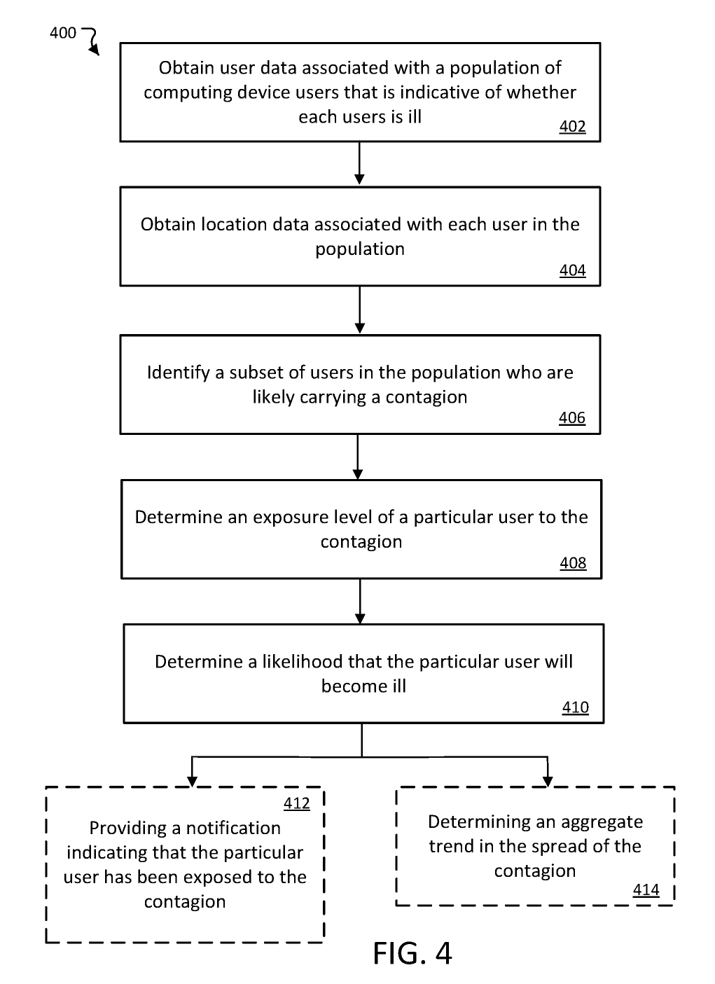

FIG. 4 depicts a flowchart of an example process 400 for predicting the spread of contagions in accordance with implementations of the present disclosure. In some implementations, the process 400 can be provided as one or more computer-executable programs executed using one or more computing devices. In some examples, process 400 is executed by one or more machine learning models. In some examples, the process 400 is executed by a CTE such as contagion tracking engine 120 of server system 102 of FIG. 1.

The system obtains user data associated with a population of computing device users (402). The user data is indicative of whether each of the users is ill or has been in contact or proximity with another user that is ill. For example, the user data can include, but is not limited to, internet search data such as search logs, user voice data, user biometric data, user motion data, and data indicating changes in a user's routine.

The system obtains location data associated with each user in the population (404). The location data can include, for example, GPS data, WiFi location data, or cellular location data. For example, the system can obtain user location data from user computing devices or from location data logs. In some examples, the system may limit its data intake to user data and location data from user computing devices that are associated with users who have “opted-in” to a contagion tracking system by, for example, providing an associated application for user download.

The system identifies a subset of the users in the population who are likely carrying a contagion (e.g., an illness) (406). For example, the system can identify a subset of potentially infected users based on the user data. As described above, the system can process the user using a machine learning model to identify indications that a user is ill. For example, the system may determine that a unique user of the population of users likely has the flu in response to identifying, within search logs, that the user has spent an hour viewing webpages that discuss flu symptoms and treatments. The system can then identify that user as likely to be carrying the flu virus.

The system determines an exposure level of a single user to the contagion (408). For example, the system can determine an exposure level of a a single user based on location data. The system can correlate location data from the single user with location data of those users who have been identified to be likely carrying the contagion. The extent to which the single user has been exposed to potentially contagious users from the population of users can be represented by an exposure level. As an example, an exposure level associated with the single user can represent a measure of the exposure that the single user has had with potentially contagious users based on the number of times the single user has been exposed to potentially contagious users, the amount of time the single user has been exposed to potentially contagious users, or a combination thereof. For example, the system can determine an exposure level for the single user based on exposure factors including, but not limited to, the number of exposures each user has with potentially infected users, the duration of time that each user was exposed to a potentially infected user, the elapsed time between exposures to potentially infected users, or a combination thereof. As another example, the system can determine specific exposure levels for each user of a subset of multiple users (e.g., a subset of unique users from among a population of registered users) based on exposure factors (as listed above) that are specific to each of the unique users in the subset.

The system can determine a likelihood that the single user will become ill (410). For example, the system can determine whether the single user is likely to become ill based on the single user's exposure level (e.g., as determined in step 408). For example, the system can identify that the single user is likely to become ill if the user's exposure level exceeds a threshold exposure value. For example, the system can compare the single user's exposure level to the threshold exposure value at regular intervals, when the exposure level changes, or both. As another example, the system can determine whether multiple users (e.g., a subset of unique users from among a population of registered users) are likely to become ill by comparing each unique user's exposure level to the threshold exposure value at regular intervals, when the exposure level changes, or both.

The system can, optionally, provide a notification to the single user that indicates the user has been exposed to the contagion and is likely to become ill (412). For example, in response to determining that the single user is likely to become ill, the system can transmit a sickness notification to the single user's computing device to inform the user that she has been exposed to the contagion and is likely to become ill. The sickness notification can include, but is not limited to, a mobile application notification, an SMS message, an e-mail, or any combination thereof. The sickness notification can include recommended preventative actions that the user can take to prevent becoming ill. For example, the notification may suggest getting a vaccination or taking immune system boosting vitamins.

The system can, optionally, determine an aggregate trend in the spread of the contagion (414). For example, the system can determine a trend in the spread of the contagion based on a plurality of exposure levels associated with a plurality of users. The system can aggregate the individual predictions of multiple users to identify trends in the spread of the contagion. For example, based on an aggregation of individual predictions, the system can determine if more or fewer people are becoming ill, whether the disease is spreading, and where the disease is spreading. The system can send appropriate notifications to authorities to enable said authorities to take actions to halt or mitigate emerging epidemics.

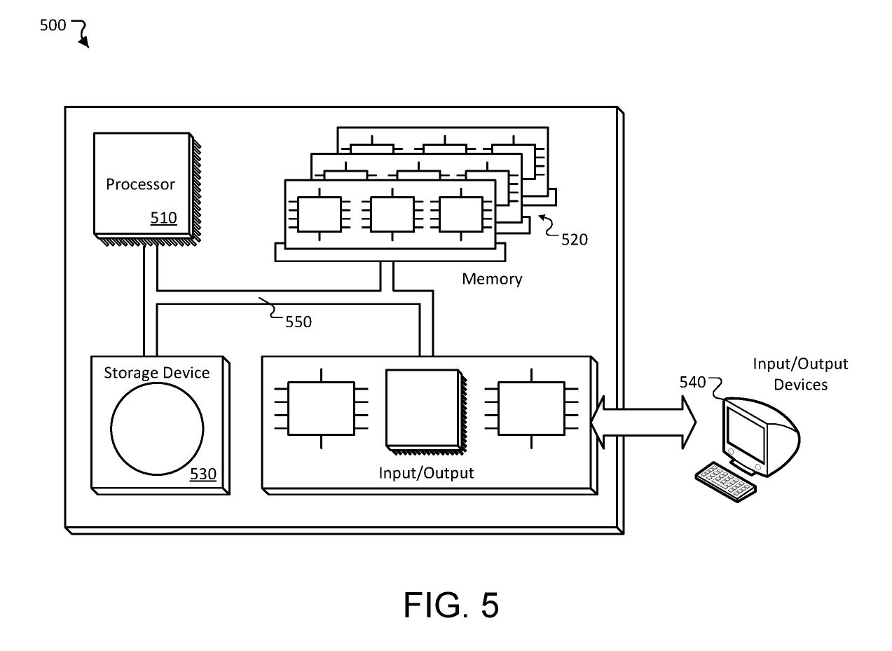

FIG. 5 is a schematic diagram of a computer system 500. The system 500 can be used to carry out the operations described in association with any of the computer-implemented methods described previously, according to some implementations. In some implementations, computing systems and devices and the functional operations described in this specification can be implemented in digital electronic circuitry, in tangibly-embodied computer software or firmware, in computer hardware, including the structures disclosed in this specification (e.g., system 500) and their structural equivalents, or in combinations of one or more of them. The system 500 is intended to include various forms of digital computers, such as laptops, desktops, workstations, personal digital assistants, servers, blade servers, mainframes, and other appropriate computers, including vehicles installed on base units or pod units of modular vehicles. The system 500 can also include mobile devices, such as personal digital assistants, cellular telephones, smartphones, and other similar computing devices. Additionally, the system can include portable storage media, such as, Universal Serial Bus (USB) flash drives. For example, the USB flash drives may store operating systems and other applications. The USB flash drives can include input/output components, such as a wireless transducer or USB connector that may be inserted into a USB port of another computing device.

The system 500 includes a processor 510, a memory 520, a storage device 530, and an input/output device 540. Each of the components 510, 520, 530, and 540 are interconnected using a system bus 550. The processor 510 is capable of processing instructions for execution within the system 500. The processor may be designed using any of a number of architectures. For example, the processor 510 may be a CISC (Complex Instruction Set Computers) processor, a RISC (Reduced Instruction Set Computer) processor, or a MISC (Minimal Instruction Set Computer) processor.

In one implementation, the processor 510 is a single-threaded processor. In another implementation, the processor 510 is a multi-threaded processor. The processor 510 is capable of processing instructions stored in the memory 520 or on the storage device 530 to display graphical information for a user interface on the input/output device 540.

The memory 520 stores information within the system 500. In one implementation, the memory 520 is a computer-readable medium. In one implementation, the memory 520 is a volatile memory unit. In another implementation, the memory 520 is a non-volatile memory unit.

The storage device 530 is capable of providing mass storage for the system 500. In one implementation, the storage device 530 is a computer-readable medium. In various different implementations, the storage device 530 may be a floppy disk device, a hard disk device, an optical disk device, or a tape device.

The input/output device 540 provides input/output operations for the system 500. In one implementation, the input/output device 540 includes a keyboard and/or pointing device. In another implementation, the input/output device 540 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

For convenience, implementations of the present disclosure have been discussed in further detail with reference to an example medical context. More specifically, the example context includes predicting the spread of a contagion (e.g., an illness). It is appreciated, however, that implementations of the present disclosure can be realized in other appropriate contexts (e.g., predicting the spread of ideas, social trends, word-of-mouth advertising, etc.).

What is claimed is:

1. A computer-implemented contagion prediction method executed by a computing system and comprising:
obtaining internet search data, the search data indicating internet searches performed by a population of users;
obtaining location data associated with each user in the population, the location data representing one or more geographic locations of each user over a period of time;
identifying, based on the search data, a subset of the population who are likely carrying a contagion;
determining, by the computing system, an exposure level of a user to the contagion based on a correlation of a first location data associated with the user with a second location data associated with one or more users in the subset of the population who are likely carrying the contagion;
determining, by the computing system and based on the exposure level, whether the user is likely to be or become ill; and
providing, for display on a user computing device, a notification indicating that the user has been exposed to the contagion.

2. The method of claim 1, further comprising determining a trend in a spread the contagion based on aggregating predictions for a plurality of individual users.

3. The method of claim 1, further comprising identifying an action for avoiding exposure to the contagion; and
providing, to a computing device associated with the user, a notification alerting the user to the action.

4. The method of claim 1, wherein determining the exposure level of the user to the contagion comprises determining that the user was present in a geographic region within an exposure window.

5. The method of claim 4, further comprising obtaining environmental data for the geographic region, wherein the exposure window for the geographic region is based, at least in part, on the environmental data.

6. The method of claim 1, wherein determining the exposure level of the user to the contagion comprises using a geographic grid to compare the first location data with the second location data.

7. The method of claim 1, wherein determining the exposure level of the user to the contagion comprises using a semantic map to compare the first location data with the second location data.

8. The method of claim 1, wherein identifying the subset of the population who are likely carrying by the contagion comprises identifying a class of the contagion.

9. The method of claim 1, wherein the internet search data includes internet search logs.

10. The method of claim 1, wherein the internet search logs include one or more annotations indicating topics described in search results, topic weightings, and an amount of time a user spent viewing one or more of the search results.

11. The method of claim 1, further comprising obtaining additional user information, wherein identifying the subset of the population who are likely carrying the contagion comprises identifying the subset based on the internet search data and the additional user information.

12. The method of claim 11, wherein the additional user information includes one or more of: user voice data, user biometric data, user motion data, or data indicating changes in a user's routine.

13. A system comprising:
one or more computers; and one or more data stores coupled to the one or more computers having instructions for executing one or more machine learning models to predict a spread of contagions to individuals stored thereon which, when executed by the one or more computers, causes the one or computers to perform operations comprising:
obtaining internet search data, the search data indicating internet searches performed by a population of users;
obtaining location data associated with each user in the population, the location data representing one or more geographic locations of each user over a period of time;
identifying, based on the search data, a subset of the population who are likely carrying a contagion;
determining an exposure level of a user to the contagion based on a correlation of a first location data associated with the user with a second location data associated with one or more users in the subset of the population who are likely carrying the contagion;
determining, based on the exposure level, whether the user is likely to be or become ill; and
providing, for display on a user computing device, a notification indicating that the user has been exposed to the contagion.

14. The system of claim 13, wherein the operations further comprise determining a trend in a spread the contagion based on aggregating predictions for a plurality of individual users.

15. The system of claim 13, wherein the operations further comprise identifying an action for avoiding exposure to the contagion; and
providing, to a computing device associated with the user, a notification alerting the user to the action.

16. The system of claim 13, wherein determining the exposure level of the user to the contagion comprises determining that the user was present in a geographic region within an exposure window.

17. The system of claim 16, wherein the operations further comprise obtaining environmental data for the geographic region, wherein the exposure window for the geographic region is based, at least in part, on the environmental data.

18. The system of claim 13, wherein determining the exposure level of the user to the contagion comprises using a geographic grid to compare the first location data with the second location data.

19. The system of claim 13, determining the exposure level of the user to the contagion comprises using a semantic map to compare the first location data with the second location data.

20. A non-transitory computer readable storage device storing instructions that, when executed by a computing system, cause the computing system to perform operations comprising:
obtaining internet search data, the search data indicating internet searches performed by a population of users;
obtaining location data associated with each user in the population, the location data representing one or more geographic locations of each user over a period of time;
identifying, based on the search data, a subset of the population who are likely carrying a contagion;
determining an exposure level of a user to the contagion based on a correlation of a first location data associated with the user with a second location data associated with one or more users in the subset of the population who are likely carrying the contagion;
determining, based on the exposure level, whether the user is likely to be or become ill; and
providing, for display on a user computing device, a notification indicating that the user has been exposed to the contagion.
